(19) United States
(12) Patent Application Publication
(10) Pub. No.: US 2016/0058866 A1
(43) Pub. Date: Mar. 3, 2016

(54) ALTERNATIVE SOLUTIONS FOR THE ADMINISTRATION OF CANNABIS DERIVED BOTANICAL PRODUCTS

(71) Applicants: Ronald D. SEKURA, Key Largo, FL (US); Roscoe M. MOORE, Rockville, MD (US); Todd M. LARABEE, Denver, CO (US)

(72) Inventors: Ronald D. SEKURA, Key Largo, FL (US); Roscoe M. MOORE, Rockville, MD (US); Todd M. LARABEE, Denver, CO (US)

(21) Appl. No.: 14/843,531

(22) Filed: Sep. 2, 2015

Related U.S. Application Data
(60) Provisional application No. 62/044,802, filed on Sep. 2, 2014.

(51) Int. Cl. A61K 47/32; A61K 31/05; A61K 9/70; A61K 31/352; A61K 9/00

(52) U.S. Cl. CPC A61K 47/32; A61K 31/352; A61K 9/006; A61K 9/70; A61K 31/05

(57) ABSTRACT

Compositions comprising: (a) at least one polyvinylpyrrolidone/vinyl acetate copolymer that forms an open matrix network; and (b) at least one ingredient tetrahydrocannabinol and cannabidiol present within the open matrix network.

### CROSS-REFERENCE TO RELATED APPLICATIONS

This application claims the benefit of U.S. Provisional Application No. 62/044,802, filed on Sep. 2, 2014, which is herein incorporated by reference in its entirety.

### FIELD OF THE INVENTION

This invention relates to compositions that comprise a Cannabis derived botanical drug product, where the compositions are, in preferred embodiments, compositions in a form suitable for oral use, including buccal and sublingual delivery and pulmonary mucosa delivery. This invention also relates to methods of making the compositions, and methods of using the compositions.

### BACKGROUND OF THE INVENTION

The botanical genus Cannabis includes the species indica and sativia. Within these species multiple distinct varieties and strains have been and continue to be developed. The genus is known to produce more than 480 different chemical substances. Among these chemicals approximately 80 distinct entities exist which are classified as cannabinoids (see e.g., Razdan R K. Structure-activity relationships in cannabinoids. Pharmacol Rev 1986; 38: 75-149, incorporated herein by reference). The two species differ in the amount of tetrahydrocannabinol (THC) produced. Cannabis sativia produces higher levels of THC. The psychotropic affects associated with THC have made the sativia species preferred as a recreational substance and for medicinal use where the psychotropic effect is desired. Not-with-standing THC and other chemical entities produced by the sativia species have significant and diverse pharmacological action.

The indica species is cultivable in cooler climates and produce more cannabidiol (CBD) than THC. This allows production of extracts with low THC. The seeds from Cannabis species are used to produce hemp oil which is used industrially and also as a nutritional supplement.

Cannabis derived materials which may contain THC and CBD in addition to numerous natural substances produced by the plants have been reported to have diverse pharmacological activities that include analgesic, anti-inflammatory, anti-cancer, antibiotic, and anti-oxidant activity.

In most jurisdictions throughout the world, including the United States, cannabinoids (which include THC, structurally related compounds, and in some instances CBD) are controlled substances and use for medical purposes has been discouraged. Since some products derived from cannabis species are economically important (e.g. hemp oil) maximum levels for cannabinoids in products have been set. In Canadian hemp seed oil THC levels are usually below detection limit of 4 ppm (parts per million, or 4 mg/kg). Legal limit for THC content in foodstuffs in Canada is 10 ppm. Some European countries have limits of 5 ppm or none-detected, some EU countries do not have such limits at all.

Relaxation of laws limiting the use of marijuana and thus cannabinoid containing products, in some jurisdictions (e.g. as of 2015 medical marijuana is considered legal in 23 states and the District of Columbia, four states having legalized recreational use) has opened the door to encourage development of new cannabinoid containing products. To a large extent major focus has been directed at systemically administered formulations and formulations taking advantage of psychotropic activities or use of cannabinoids as antioxidants and neuroprotectants.

U.S. Pat. No. 6,630,507, incorporated herein by reference, discloses pharmaceutical compounds and compositions that are useful as tissue protectants, such as neuroprotectants and cardioprotectants. The compounds and compositions are disclosed to be used in the treatment of acute ischemic neurological insults or chronic neurodegenerative diseases. The disclosed compositions include cannabidiol and other cannabinoids, and the compositions are disclosed to include THC in amounts that do not promote psychoactive or psychotoxic effects. Accordingly, there is no disclosure of oral sublingual or buccal compositions that include THC in amounts that exceed the detection limit of THC.

In the current disclosure we direct our claims to the delivery of cannabis derived botanical products for medical and recreational products that use sublingual and buccal formulations. Sublingual formulations involve placing the product under the tongue where it dissolves and the active substance enters the body by absorption through the oral mucosal membrane. Buccal administration involves placing the product between the cheek and the gum. Here it dissolves and is absorbed through the oral mucosal membrane. Several options are available for delivery by these routes. They include liquid formulations and sprays. United States Patent Application 2012/0328718 describes a sublingual spray as a method for delivering cannabis derived products. In the current instance we prefer oral wafers, oral gels, oral lozenges, oral quick disintegrating tablets, orally dissolving strips, oral lyophilisates, and sublingual or buccal effervescent formulations. Each of these approaches allows for precise dosing, unit dose packaging and child resistant packaging.

Other than US 2012/0328718, there is no description of sublingual or buccal delivery of THC or CBD containing formulations. We consider the art described under US 2012/0328718 cumbersome and that the approach described here describes products that are safer and easier to administer with precise dosing.

In view of the foregoing, there is a need for oral sublingual and buccal formulations that comprise a Cannabis-derived botanical product. These embodiments can be used to deliver Cannabis derived botanical product for both medicinal and recreational purposes. Delivery by the route disclosed herein will eliminate problems associated with smoke free legislation and the approach can be used to treat the spectrum of conditions where Cannabis derived botanical products is believed to be of benefit.

### BRIEF SUMMARY OF THE INVENTION

One object of the invention is to provide an oral, buccal, or sublingual preparation comprising a Cannabis derived botanical drug product such that single doses of one of THC or CBD or both is delivered at between 1 and 30 milligrams per single dose. Other objects of the invention include methods of making the oral sublingual or buccal preparations and methods of using the oral sublingual or buccal preparations for treating diseases and other medical conditions and also for recreational purposes.

### BRIEF DESCRIPTION OF THE DRAWINGS

Not Applicable

### EMBODIMENTS OF THE PRESENT INVENTION

1. A composition comprising: (a) at least one polyvinylpyrrolidone/vinyl acetate copolymer that forms an open matrix network; and (b) at least one of tetrahydrocannabinol and cannabidiol present within the open matrix network.
2. The composition according to embodiment 1, wherein at least 80% of the composition dissolves within 30 seconds upon contact with an aqueous solution or with saliva, and the composition is an orodispersible pharmaceutical dosage form.
3. The composition according to embodiment 1, wherein tetrahydrocannabinol is present within the open matrix network.
4. The composition according to embodiment 1, wherein cannabidiol is present within the open matrix network.
5. The composition according to embodiment 1, wherein tetrahydrocannabinol and cannabidiol are present within the open matrix network.
6. The composition according to embodiment 1, wherein said at least one of tetrahydrocannabinol and cannabidiol is present within the open matrix network in an amount of from 1 to 30 milligrams per single dose.
7. The composition according to embodiment 1, wherein tetrahydrocannabinol is present within the open matrix network and present in an amount of from 1 to 30 milligrams per single dose.
8. The composition according to embodiment 1, wherein cannabidiol is present within the open matrix network and present in an amount of from 1 to 30 milligrams per single dose.
9. The composition according to embodiment 1, wherein tetrahydrocannabinol and cannabidiol are present within the open matrix network and present in an amount of from 1 to 30 milligrams per single dose.
10. The composition according to embodiment 1, wherein said least one of tetrahydrocannabinol and cannabidiol is present within the open matrix network in an amount of from 5 to 10 milligrams per single dose.
11. The composition according to embodiment 1, wherein tetrahydrocannabinol is present within the open matrix network and present in an amount of from 5 to 10 milligrams per single dose.
12. The composition according to embodiment 1, wherein cannabidiol is present within the open matrix network and present in an amount of from 5 to 10 milligrams per single dose.
13. The composition according to embodiment 1, wherein tetrahydrocannabinol and cannabidiol are present within the open matrix network and present in an amount of from 5 to 10 milligrams per single dose.

### DETAILED DESCRIPTION OF THE INVENTION

Unless indicated otherwise, the indefinite articles “a” and “an” are synonymous with “at least one” or “one or more.” Unless indicated otherwise, definite articles used herein, such as “the,” also include the plural of the noun. The units for the amounts of compounds in oral, sublingual, or buccal formulations as used herein are typically represented as milligrams per single dose, unless otherwise indicated. The terms “comprising,” “consisting essentially of,” “consisting of,” and their related forms (e.g. comprises, etc.), have their ordinary and customary meaning under U.S. patent law and are within the scope of the present invention. Unless otherwise indicated, the elements of methods or processes described herein are usually performed in the order in which they are listed.

Unless otherwise indicated, the term “Cannabis” used herein refers to at least one of Cannabis sativia and Cannabis indica, and other species and subspecies in the Cannabis genus. Some of the materials which are produced by the Cannabis species have been shown to have pharmacologic activity. Such materials are discussed in turn.

Tetrahydrocannabinol, which is abbreviated herein as “THC” unless otherwise indicated, is the principal psychoactive constituent (or cannabinoid) of the cannabis plant. THC is also known as delta-9-tetrahydrocannabinol (Δ9-THC). THC was first isolated in 1964, and, in its pure form, it is a glassy solid when cold and becomes viscous and sticky if warmed. Pharmaceutical formulations that comprise THC, known by its INN dronabinol, are available by prescription in the U.S. and Canada under the brand name MARINOL. THC is an aromatic terpenoid, and it has a very low solubility in water but good solubility in most organic solvents, specifically lipids and alcohols. THC also exhibits high UV-B (280-315 nm) absorbance.

Cannabidiol (CBD) is one of at least 85 cannabinoids found in cannabis. It is a major constituent of the plant, second to THC, and represents up to 40% in its extracts. Compared with THC, cannabidiol is non-psychoactive, and is considered to have a wider scope of medical applications than THC, including to epilepsy, multiple sclerosis spasms, anxiety disorders, schizophrenia, nausea, convulsion and inflammation, as well as inhibiting cancer cell growth. CBD may decrease the rate of THC clearance from the body, perhaps by interfering with the metabolism of THC in the liver. CBD has displayed sedative effects in animal tests, while other studies have found that CBD may increases alertness. CBD has been shown to reduce growth of aggressive human breast cancer cells in vitro, and to reduce their invasiveness.

CBD is an anti-oxidant, has anti-inflammatory activity and analgesic properties in animal studies. It has been shown to inhibit the growth of bacteria, and is thought to exhibit psychoactive properties that are distinct from THC that include anticonvulsant and anti-epileptic properties.

Other pharmacologically active cannabinoid derived materials include β-sitosterol, tocopherols, terpenes, methyl salicylate, hemp oil or hempseed oil, hash oil, and hashish. These are discussed in turn.

Beta-Sitosterol: Although studies have primarily demonstrated the efficacy of β-sitosterol in reducing hypercholesterolemia, additional antiviral, antifungal, and anti-inflammatory properties have been studied and observed.

Tocopherols: Antioxidant properties of alpha and gamma tocopherols have been known and exploited for some time.

Terpenes: pharmacological properties of β-caryophyllene would include anti-inflammatory/and cytoprotective activities which may too be active in the seed oil.

Methyl salicylate: a compound that exhibits antipyretic, anti-inflammatory and analgesic properties.

Hemp oil or hempseed oil is obtained by pressing hemp seeds. Cold pressed, unrefined hemp oil is dark to clear light green in color, with a pleasant nutty flavor. The darker the color, the grassier the flavor. While most hemp oil is produced from strains of Cannabis sativia that produce low levels of THC, we can include in some of our formulations hemp oil from strains of Cannabis indica.

Refined hempseed oil is clear and colorless, with little flavor and lacks natural vitamins and antioxidants. Refined hempseed oil is primarily used in body care products. Industrial hempseed oil is used in lubricants, paints, inks, fuel, and plastics. Hempseed oil has found some limited use in the production of soaps, shampoos and detergents. The oil is of high nutritional value because of its 3:1 ratio of omega-6 to omega-3 essential fatty acids, which matches the balance required by the human body. It has also received attention in recent years as a possible feedstock for the large-scale production of biodiesel. There are a number of organizations that promote the production and use of hempseed oil.

Hempseed oil is generally manufactured from varieties of Cannabis sativa that do not contain significant amounts of THC, the psychoactive element present in the cannabis plant. This manufacturing process typically includes cleaning the seed to 99.99% before pressing the oil. There is no THC within the hempseed, although trace amounts of THC may be found in hempseed oil when plant matter adheres to the seed surface during manufacturing. The modern production of hempseed oil, particularly in Canada, has successfully lowered THC values since 1998. Regular accredited sampling of THC in Canadian hemp seed oil shows THC levels usually below detection limit of 4 ppm (parts per million, or 4 mg/kg). Legal limit for THC content in foodstuffs in Canada is 10 ppm. Some European countries have limits of 5 ppm or none-detected, some EU countries do not have such limits at all. For some products production of hemp oil from Cannabis species and strains that produce THC is desirable. Hemp oil is of high nutritional value because of its 3:1 ratio of omega-6 to omega-3 essential fatty acids,

About 30-35% of the weight of hempseed is an edible oil that contains about 80% as essential fatty acids (EFAs); i.e., linoleic acid, omega-6 (LA, 55%), α-linolenic acid, omega-3 (ALA, 22%), in addition to γ-linolenic acid, omega-6 (GLA, 1-4%) and stearidonic acid, omega-3 (SDA, 0-2%). The proportions of linoleic acid and α-linolenic acid in one tablespoon per day (15 ml) of hempseed oil easily provide human daily requirements for EFAs. Unlike flaxseed oil, hempseed oil can be used continuously without developing a deficiency or other imbalance of EFAs. This has been demonstrated in a clinical study, where the daily ingestion of flaxseed oil decreased the endogenous production of GLA.

Hash oil, not to be confused with hempseed oil, is used for both medicinal and recreational purposes and made from the mature female flowers and leaves of the drug cannabis, thus having a much higher THC content. Hash oil should not be confused with hemp, as the modern usage of the word ‘hemp’ is reserved for plants that meet the legal requirement of containing 0.3% THC or less.

Hash oil (also known as honey oil, dabs, shatter, or earwax) is a resinous matrix of cannabinoids obtained from the cannabis plant by solvent extraction. Hash oil is the most potent of three main cannabis products, which are herb (marijuana), resin (hashish), and oil (hash oil).

Reported THC contents vary between sources. The 2009 World Drug Reports states that the THC content “may exceed 60%”. A 2013 American forensic science book gave a range of 10-30% delta-9 THC by weight. A 1972 American forensic journal reported a range of 20-65%.

Hash oil is a cannabis product obtained from separating resins from leaves by solvent extraction. Cannabis is boiled in a solvent to form a viscous liquid which is then strained and the solvent is evaporated to yield hash oil. Flammable solvents used in extraction makes the process dangerous. Newer methods like CO₂ extraction provide a safer way to extract the resin. CO₂ extraction is a method of using high pressure to force a solvent through plant matter. The solvent used for extraction is carbon dioxide. The solvent is pushed through the plant matter at a high pressure and separates the cannabinoid resins and terpenes from the plant matter. The result is pure, transparent, amber oil. Carbon dioxide is a natural product which leaves behind no residues. The purity of CO₂ is its biggest advantage over all other solvents used for plant extraction. Currently, a popular extraction solvent is butane which can potentially leave heavy metals behind in the extracted product.

Hashish, often known as “hash”, is a cannabis product composed of compressed or purified preparations of stalked resin glands, called trichomes, collected from the unfertilized buds of the cannabis plant. It contains the same active ingredients—such as THC and other cannabinoids—but in higher concentrations than unsifted buds or leaves.

Hashish may be solid or resinous depending on the preparation; pressed hashish is usually solid, whereas water-purified hashish—often called “bubble melt hash”—is often a paste-like substance with varying hardness and pliability, its color most commonly light to dark brown but varying toward green, yellow, black or red. It is consumed by being heated in a pipe, hookah, bong, bubbler, vaporizer, hot knife (placed between the tips of two heated knife blades), smoked in joints, mixed with cannabis buds or tobacco (the latter being more common in Europe, South America as Brazil and Africa), or cooked in foods. Hashish use as a medicine and recreational drug dates back to at least the 3rd millennium BC.

With a view to the foregoing, the present inventors have formulated compositions that comprise a Cannabis derived botanical drug product. The US Food and Drug Administration has defined Botanical Drug Product as follows:
A botanical drug product consists of vegetable materials, which may include plant materials, algae, macroscopic fungi, or combinations thereof; or
A botanical drug product may be available as (but not limited to) a solution (e.g., tea), powder, tablet, capsule, elixir, topical, or injection.

Botanical drug products often have unique features, for example, complex mixtures, lack of a distinct active ingredient, and substantial prior human use. Fermentation products and highly purified or chemically modified botanical substances are not considered botanical drug products.

Systematic delivery of cannabis derived products for both recreational and medical purposes in the past has been achieved largely by smoking or ingestion. There have been descriptions of delivery by additional paths such as aerosols, suppositories, transdermal patches, and sublingual adsorption from cannabis tinctures and aerosols. Problems with each of these delivery systems include discomfort with inhaling smoke and calibration of the dose delivered by smoke inhalation. With regard to ingestion, there can be a time delay before effects are observed, which can lead to overdosing. These problems can be addressed by aerosols (e.g. electronic cigarettes), but not all users are comfortable with aerosol use, and the use of aerosols as well as tinctures can result in difficulty in controlling the amount of the product delivered to the user.

In view of the foregoing, the present invention relates to compositions that comprise a Cannabis derived botanical drug product, where the compositions are, in preferred embodiments, compositions in a form suitable for oral sublingual or buccal or pulmonary aerosol use. In addition the invention relates to individual cannabinoids or mixtures of cannabinoids that are isolated from cannabis or prepared synthetically. The cannabinoids described by: Razdan R K. Structure-activity relationships in cannabinoids. Pharmacol Rev 1986; 38: 75-149 (incorporated herein by reference) can be used. This invention also relates to methods of making the compositions, and methods of using the compositions.

The form suitable for oral use, unless otherwise indicated, relates to sublingual oral compositions such as oral lysophilisates or buccal mucosa oral compositions. The sublingual oral compositions or buccal mucosa oral compositions are preferably in solid form or semi-solid form. The solid form or semi-solid form can be, preferably, an oral wafer, an oral gel, an oral lozenge, an oral fast dissolving tablet, an orally dissolving strip, an oral lysophilisates, a buccoadhesive formulation, and/or a sublingual or buccal effervescent tablet.

Without wishing to be bound to a particular theory, it is believed that the form suitable for oral use is sufficient to deliver a Cannabis derived biological drug product to the user with a greater degree of precision and accuracy as to dosage amounts. It is also believed that the use of the form suitable for oral use increases the absorbance rate of the Cannabis derived biological drug product through the mucosal surface and into the bloodstream. The use of the form suitable for oral use can also have the advantage of avoiding the inhalation of other agents that may be present when the Cannabis derived biological drug product is in cigarette or aerosol form.

As used herein and unless otherwise indicated, the term “Cannabis derived biological drug product” is prepared form varieties of Cannabis indica and sativia, which include oils pressed from the seeds; powders prepared for various plant parts including flowers, leaves, stems, buds, and trichomes; extracts prepared from the various parts including flowers, leaves, stems, buds, and trichomes, which includes extracts prepared using organic solvents, water extraction, alcohol extracts, and liquid carbon dioxide extracts; hemp oil; hashish oil; hashish; and fractions of extracts or oils prepared by chromatography, phase partition, temperature fractionation, distillation or other methods employed for fractionation.

Most preferably, the term “a Cannabis derived botanical drug product” refers to the compounds obtained from chemical extraction of Cannabis, provided that at least one of THC and CBD is present in the Cannabis derived botanical drug product.

In the present invention, the term “oral lyophilisate” usually denotes a pharmaceutical composition which is lyophilized (i.e. which has undergone successive steps of freezing, drying and sublimation under reduced pressure) and which is intended for oral administration. These oral lyophilisates are most commonly in the form of dry and porous tablets obtained by lyophilization, which disintegrate very rapidly as soon as they are brought into contact with saliva. These tablets may contain one or more pharmaceutical active ingredients. These oral lyophilisates can also be in the form of a thin strip, also obtained by lyophilization. The dimensions of the dry and porous tablets and thin strips are not particularly limited so long as they are in a size suitable for oral use.

The term “spray-drying” will be used in the present application to describe the process of dehydration which makes it possible to go from a liquid form (solution, suspension) to a dry form (powder) by pulverization. This process comprises pulverizing a solution or a suspension in the form of droplets or of fine particles in an environment which allows rapid drying thereof through evaporation of the solvent under the effect of heat optionally combined with a stream of air or of another

The term “absorption” denotes the passing of an active ingredient from the external medium (saliva, gastrointestinal fluid) to the bloodstream.

In the present invention, the term “bioavailability” will be used to describe the fraction of active ingredient that is actually absorbed by the organism and reaches the bloodstream, relative to the dose of medicament contained in the pharmaceutical composition administered.

In the present invention, the terms “copovidone or copolyvidone” usually denotes the vinyl acetate/polyvinylpyrrolidone copolymer. This water-soluble copolymer is sold in particular under the trade names KOLLIDON VA, PLASDONE or LUVISKOL. The particular grade sold under the name KOLLIDON VA 64 can be obtained by polymerization of 6 parts of polyvinylpyrrolidone with 4 parts of vinyl acetate.

In the present invention, the term “dissolution” usually describes the changing from the solid state to the state dissolved in water of a medicament, and more particularly of the active ingredient(s) that it contains; the dissolution can be quantified by quantitative determination (dissolution test). In the invention, the term “dissolution” will be used as a synonym of the term “solubilization” which corresponds to the visible change of a solid to the dissolved state (absence of particles in the solution).

The terms “low solubility” or “with low solubility” or “with low water-solubility” denote, in the present invention, all active ingredients of which the solubility in water is defined as low to zero by the United States pharmacopeia (USP 32) according to the amount of water necessary for the dissolution of one part of solute:

Low solubility: 100 to 1000 parts of water necessary for dissolution of one part of solute.

Very low solubility: 1000 to 10 000 parts of water necessary.

Virtually insoluble: more than 10 000 parts of water necessary.

The term “microemulsion” usually denotes an emulsion obtained by using at least two nonionic surfactants including at least one main surfactant and one second “cosurfactant,” acting in combination with said main surfactant. Such a microemulsion has great stability and the globules of the emulsion are very small in size: of the order of a few nanometers to a few micrometers.

In the present invention, the term “wettability” usually denotes the ability of a liquid to remain in contact with a solid when these two elements are brought together. The degree of wettability is the result of the cohesive forces exerted on the liquid which oppose the spreading of said liquid on the surface of the solid, and of the adhesive forces that are exerted from the solid onto the liquid, which promote spreading of the latter. The chemical composition of the liquid and the chemical nature of the solid can therefore influence the wettability. In the subsequent text, the term “wettability” usually relates to active ingredients which have low solubility or very low solubility when they are brought into contact with the aqueous liquid phase necessary for the preparation of the oral lyophilisates which are subjects of the invention.

In the context of the present invention, the term “conventional surfactants” usually denotes surfactants commonly used in the food and cosmetics industries for example, but which have not been approved for use in the pharmaceutical industry, and also the ionic surfactants approved for pharmaceutical use.

The present invention relates to lyophilized compositional forms for oral administration of active ingredients and production methods for making the lyophilized composition forms. The oral lyophilisates according to the invention are capable of improving both the dissolution and the absorption in the blood stream of soluble, low-solubility or even very-low-solubility ingredients. THC and CBD are both considered ingredients where the dissolution can be improved when present in the lyophilized compositional forms.

The lyophilized formulations can be pharmaceutical forms that may be based on a liquid phase in the form of a solution, a suspension or an emulsion, the water of which is subsequently sublimated, and may comprises various excipients such as diluents, flavors, sweeteners, and combinations thereof. These formulations have the advantage of completely and very rapidly disintegrating on contact with saliva or the aqueous medium.

Polyvinylpyrrolidone copolymers of grades having the following PVP/VA ratios: 30/70; 50/50; 70/30 and 20/80 are used in certain pharmaceutical products, such as granulating agents, spray-on bandages, or antiseptic, anesthetic or else antibiotic sprays, but they are soluble only in ethanol or isopropanol. They are water-dispersible but are not water-soluble.

Copovidone or polyvinylpyrrolidone copolymer having a PVP/VA ratio of 60/40 (6 parts of vinylpyrrolidone for 4 parts of vinyl acetate) is an excipient which is soluble in water, in isopropanol and in ethanol. The copovidone sold under the trademark KOLLIDON VA 64 and is an excellent binder for tablets or microgranules obtained by dry or wet granulation.

Polyvinylpyrrolidone/vinyl acetate copolymers, and copovidone in particular, can be in lyophilized formulations. This copolymer can be used as a filler in the composition of hair dye in powder form, obtained by lyophilization or spray-drying. In these nonpharmaceutical formulations, the copovidone is part of the composition of the resin forming the film of the dyes described in this document.

Polyvinylpyrrolidone/vinyl acetate copolymers can be used as excipients in oral formulations and oral pharmaceutical formulations.

In the present invention, the use of copovidone in lyophilized formulations obtained from a liquid aqueous phase free of organic solvent, such as the presence of organic solvent below the detection limit, and of ionic surfactant makes it possible to significantly improve both the rate of dissolution and the bioavailability of active agents of varying solubility, especially low solubility or very low solubility or which are virtually insoluble in water. As used herein, “organic solvent” denotes those solvents identified by the United States Center of Disease Control in their Criteria Documents and Current Intelligence Bulletins. Thus, this term does not encompass solvents such as water, methanol, ethanol, etc.

In addition, the oral lyophilisates according to the invention have the advantage of being pH-independent insofar as the polyvinylpyrrolidone/vinyl acetate copolymer is nonionic. The lyophilized formulations according to the invention have the advantage of being able to be used for the formulation of active agents exhibiting very low concentrations.

Embodiments of the present invention relate to oral compositions in oral lyophilisate forms, intended to improve the wettability, the hydrophilization and the solubilization of low-solubility active ingredients with the aim of improving the bioavailability thereof.

Embodiments of the present invention also relates to the process for producing these oral compositions. A feature of the pharmaceutical formulations in accordance with the invention is that they are produced from a liquid aqueous phase free of organic solvent and of ionic surfactant, comprising at least one polyvinylpyrrolidone/polyvinyl acetate copolymer, the aqueous phase being subjected to a subsequent lyophilization step. Said polyvinylpyrrolidone/polyvinyl acetate copolymer has a PVP/VA molar ratio of 60/40.

The oral lyophilisates in accordance with the invention are usually obtained after sublimation of the water contained in a liquid aqueous phase free of organic solvent and of ionic surfactant, comprising at least one polyvinylpyrrolidone/polyvinyl acetate (PVP/VA) copolymer having a PVP/VA ratio of 60/40. This copolymer is obtained by polymerization of units of vinylpyrrolidone (PVP) and units of vinyl acetate (VA).

In the present invention, this copolymer has a PVP/VA ratio of 60/40 which gives it a water-solubility that allows it to be distributed homogeneously around the active ingredient during the formation of the aqueous liquid phase, whether the active ingredient is dissolved in the form of particles (suspension, dispersion) or else dissolved in a lipid solvent present in the aqueous phase in the form of micro-droplets (emulsion).

This copolymer, which results from polymerization of 6 parts of vinylpyrrolidone with 4 parts of vinyl acetate, is sold, for example, under the grade KOLLIDON VA 64 (BASF) or under the grade PLASDONE 5630 (ISP). The PVP/VA copolymer is present in the oral lyophilisates in an amount of from 0.1% and 80% by weight relative to the dry weight of the oral lyophilisate. Preferentially, the proportion of copovidone is between 1% and 65% by weight relative to the total dry weight of the oral lyophilisate, even more preferentially between 5% and 40% by weight.

The proportion of PVP/VA copolymer in the liquid aqueous phase of the formulations of the invention advantageously represents between 5% and 20% by weight relative to the total weight of the liquid phase.

The oral lyophilized tablets in accordance with the invention may contain one or more active ingredients which represent(s), as appropriate, between 0.1% and 60% by weight relative to the total weight of the lyophilisates. Preferentially, the proportion of the active ingredient (s) in the lyophilized tablets of the invention is between 1% and 40%.

Thus, the active ingredient/copovidone proportion of the lyophilisates of the invention ranges between 1:800 and 600:1. Preferentially, the active ingredient/copovidone proportion is between 1:40 and 8:1, even more preferentially between 1:20 and 5:1.

The lyophilized formulations in accordance with the invention can advantageously comprise a very large number of active ingredients, or mixtures of ingredients, as has been described above. This is because the production process works with virtually all ingredients, with the exception of those of which the activity is sensitive to variations in temperature (in particular enzymes). Specifically, the physicochemical behavior of the active ingredient, and in particular its solubility in water, is not an obstacle to the preparation of the oral lyophilisates according to the invention since the aqueous phase which serves to bring the active ingredient into close contact with the copolymer can without distinction be in the form of a solution, a suspension, a dispersion or else an emulsion or microemulsion. The step of sublimation of the water contained in the aqueous phase is not in fact limited by the presence of dispersed solid particles (suspension) or even of lipid micro-droplets (emulsion) in which the active agent is in dissolved form. The present invention is particularly suitable for the administration of active ingredients which have low solubility or very low solubility or even which are virtually insoluble in water. As a result, active ingredients of which the water-solubility will be described as low to very low, or even active ingredients termed insoluble, may advantageously be used.

In all embodiments of the present invention, at least one of THC and CBD is present in the form suitable for use (e.g. oral lyophilisates) in an amount of from 1 to 30 milligrams per single dose, preferably from 2 to 20 milligrams per single dose, most preferably from 5 to 10 milligrams per single dose.

In some embodiments of the present invention, the oral lyophilisates of the present invention can include additional ingredients described in WO 2011/086194, incorporated herein by reference. These additional ingredients include, but are not limited to, binders, viscosity modifiers, fillers, diluents, gelling agents, and surfactants.

Specifically, with regard to the binders, the binders comprise all the water-soluble or water-dispersible substances that allow cohesion of the mass of the tablet and are pharmaceutically acceptable and inert with regard to the active ingredient(s) of interest. Preferably, the binders are polypeptides, such as gelatin, colloids, high-molecular-weight polysaccharides, large polymers capable of giving colloidal solutions, such as resins or natural gums (for example, gum Arabic, gum tragacanth) or semisynthetic gums (for example, xanthan gum or glycosylglucans), dextran, in particular the grades known as Dextran 20, 40 and 70, dextrin, alginates, in particular sodium alginate, pectinates, carboxymethylcellulose, water-dispersible starch derivatives, colloidal silicas or bentonites. Generally, the binders of the present invention represent between approximately 0.01% and up to approximately 30% of the dry mass of the final oral lyophilisate, preferentially between approximately 0.5% and 20%.

The oral lyophilisates according to the invention can further comprise at least one filler or diluents, which are excipients commonly used in lyophilized formulations.

These agents are preferentially pharmaceutically acceptable water-soluble substances such as sugars and derivatives thereof, for instance glucose, lactose, glycine, maltodextrin, isomalt, or cyclodextrins and derivatives thereof, or alcohol sugars such as mannitol, sorbitol or xylitol, for example.

Preferably, mannitol is used as filler for implementing the invention, said mannitol being used alone or in combination with another filler, for instance dextran.

The diluents may also belong to the family of oxides, for instance magnesium oxide, carbonates (for instance calcium carbonate) or phosphates (such as tricalcium phosphate). The fillers may represent between 0.5% and 90% by weight relative to the total dry weight of the oral lyophilisates. The preferred amount of diluent is between approximately 10% and approximately 50% relative to the dry weight of the lyophilized tablets.

The oral lyophilisates according to the invention may also contain, either alone or in combination with one another, sweeteners, taste-masking agents or flavors intended to increase the palatability of the medicament in the mouth. Specific mention is made of sucrose, glucose, xylose, sucralose, acesulfame, saccharin, saccharinates, cyclamates, aspartame, ammonium glycyrrhizinate or else citric acid, ascorbic acid or tartaric acid.

Generally, any other substance normally used as a taste modifier in the pharmaceutical industry and which is compatible with the active ingredient (s) used can be used for preparing the pharmaceutical compositions according to the invention.

The amount of sweetener or of taste-masking agent is generally between 0.01% and approximately 5%, preferentially between approximately 0.05% and 1% by weight relative to the dry weight of the tablets according to the invention.

Colorants and preservatives may also be used in the formulations of the invention and are those normally used in pharmaceutical formulations in general and for coloring lyophilized oral tablets in particular.

They comprise, for example: amaranth, barley extract, caramel, cochineal, carotene, copper-chlorophyll complexes, iron oxides, riboflavin, grape skin extract, titanium dioxide, erythrosine or methylene blue, for example.

In addition to the excipients mentioned above, the lyophilized formulations according to the invention may also contain, conventionally, other additional “cohesion” excipients intended, for example, to prevent breaking of the tablets. Among these excipients are, in particular, silica or hydrophilic diluents, for instance certain sugars, such as levulose for example.

The oral lyophilisates according to the invention also have an open matrix structure. In embodiments of the present invention, at least one of THC and CBD is present in the open matrix structure.

The present invention also relates to the process for producing oral lyophilisates based on PVP/VA copolymer, which are capable of improving the dissolution of low-solubility active ingredients, comprising the following stages:
a. preparing a liquid aqueous phase free of organic solvent and of ionic surfactant, containing at least one pharmaceutical active ingredient, one filler and/or one binder and the PVP/VA copolymer of which the PVP/VA ratio is 60/40;
b. stirring the aqueous phase until a homogeneous mixture is obtained;
c. distributing the resulting homogeneous mixture into a preformed cavity (for example a mold or a blister);
d. freezing the liquid phase thus distributed at a temperature of approximately −20° C. to approximately −50° C. until a frozen mixture is obtained;
e. lyophilizing said frozen mixture; and
f. optionally, carrying out a second, drying step on the resulting lyophilized mixture.

The process for preparing the lyophilisates in accordance with the invention is based on a common lyophilization process.

In a first step, an aqueous liquid phase intended to be lyophilized and containing the active ingredient (s) of interest is prepared. In accordance with the present invention, this liquid phase does not contain organic solvents.

According to the physicochemical characteristics of the active ingredient (s) used and the initial forms, structures and particle sizes thereof, a solution, suspension, emulsion or a preparation precooled to a semi-frozen consistency of sorbet type can be advantageously prepared as liquid aqueous phase. This liquid phase therefore contains at least the active ingredient(s) of interest, a binder and/or filler, the PVP/VA copolymer and water.

Advantageously, other excipients can be added to this liquid aqueous phase free of organic solvent and of ionic surfactant, for instance, and in a non-limiting manner: colorants, sweeteners, taste-masking agents or preservatives.

The mixing of the active ingredient and of the appropriate excipients including the copovidone is generally carried out in a mixer equipped with a vacuum system. When it is in solid form, the active ingredient and the excipients in powder form are mixed until an acceptable homogeneous mixture is obtained. If the active ingredient is in liquid form (for example dissolved in a lipid liquid, it is mixed in a similar manner with the excipients mentioned above, which can be in solid or liquid form without distinction.

Generally, this mixing step lasts 5 to 30 minutes under reduced pressure (generally from 100 to 300 HPa) with the aim of “degassing” the powders and allowing suctioning of the water.

The aqueous phase is then finished off by adding water to the previously formed solid or liquid mixture by suctioning. The resulting solution, suspension or emulsion is then mixed under reduced pressure (generally from 100 to 300 HPa) for a period of 30 to 90 minutes until a perfectly homogeneous mixture is obtained. The amount of water introduced so as to form the aqueous phase intended to be lyophilized is determined in such a way that this phase has acceptable rheological properties, i.e. a viscosity which allows it to have good flow, to be able to be easily mixed to give a perfectly homogeneous phase and to be able to be easily divided up and distributed uniformly into individual molds or blisters. In most cases, the amount of water will be adjusted in such a way that the solid mass constitutes approximately between 30% and 80% of the mixture. In certain preparations, this proportion can vary between 40% and 60% relative to the dry mass of the final product. In a second step, once completely homogenized, the liquid preparation obtained is distributed into the preformed cavities, generally in the form of thermoformed molds made of PVC, PVDC or aluminum foils. The distribution step is carried out mechanically, the overall volume of liquid phase being divided up into unit doses having a predetermined shape, size and volume. The amount of active ingredient (s) in the liquid phase and the shape and the size of the cavities are calculated so as to obtain a precisely defined amount of active ingredient(s) in each unit dose. The cavities (for example the blisters) containing the product are then placed on the platforms of the lyophilizer. These platforms are metal supports for the blisters, which can be easily heated or cooled and which allow an abrupt modification of the temperature of the product contained in the blisters.

Once the cavities are filled with the suitable volume of liquid preparation, the freezing step begins. It starts at very low temperature (generally between −20° C. and −50° C.) below the eutectic point (i.e. the temperature at which there is total solidification of the liquid phase) at atmospheric pressure, for approximately 30 to 90 minutes, until a solid phase is obtained.

The conventional lyophilizers that can be used are in general mechanical lyophilizers using a compressor system, such as those sold, for example, by the companies Usifroid, Virtis or BOC Edwards. At the end of this step, the molecules of active ingredient are immobilized, the therapeutic properties thereof remaining unimpaired insofar as the number of chemical reactions is virtually zero at these very low temperatures.

The temperature and pressure conditions of the freezing phase are adjusted according to the composition of the liquid phase which may, depending on the nature and the concentration of the filler and/or the binder and the copovidone, be more or less easy to freeze.

The preparation thus frozen is then subjected to sublimation, i.e. the product is dried under vacuum, passing from the solid phase to the gaseous phase in a controlled manner by applying thereto both heat and a low pressure (generally between 100 and 600 μbar), preferentially between approximately 150 and 400 μbar. The temperature of the platforms supporting the molds or blisters is taken rapidly from approximately −30/−50° C. up to approximately +40/+70° C. while the temperature of the product increases gently from the freezing point up to +30/+50° C. According to the amount of material contained in each mold, the sublimation phase lasts, in total, between 300 and 800 minutes.

Under these conditions, the ice is converted directly to vapor phase. The product resulting therefrom is a pharmaceutical formulation in the form of a porous solid which has retained its initial shape and the therapeutic qualities of the active ingredient (s). Optionally, an additional drying phase (also called secondary drying) is applied to the product and the residual water (termed “bound” water) is extracted by desorption (i.e. by evaporation of the liquid phase into the gaseous phase, contrary to sublimation, which corresponds to the evaporation of a solid phase into an gaseous phase).

This is because the presence of water in the product is often responsible for rapid degradation of the active ingredient, resulting in poor storage stability of the product. This secondary drying step is generally carried out at between 40° C. and 50° C. and at a pressure of approximately 100 μbar for a period of from 60 to 120 minutes. The average duration of secondary drying in the lyophilizer is between 60 and 90 minutes, but can advantageously be prolonged, without any negative impact on the product.

The final lyophilized tablets are then heat-sealed in their cavities, for example, in the case of blisters with aluminum foils at a temperature of between 120° C. and 160° C. for approximately 1 to 3 seconds.

The oral lyophilized formulations of the invention can thus be used for the production of medicaments comprising at least one active ingredient and in particular active ingredients which have low solubility or very low solubility or which are virtually insoluble in water.

In view of the foregoing, one embodiment of the present invention relates to composition comprisings: (a) at least one polyvinylpyrrolidone/vinyl acetate copolymer that forms an open matrix network; and (b) at least one of tetrahydrocannabinol and cannabidiol present within the open matrix network.

Another embodiment of the present invention is a method of making the compositions comprising a Cannabis derived botanical drug product, where, preferably, the compositions are formed by these methods are sublingual or buccal formulations. These methods preferably comprise selecting a plant of Cannabis as a starting material and selecting parts of the plant, where the preferred plant parts are trichomes, flowers, leaves, stems, buds or powders thereof. The most preferred plant part is trichomes. In other embodiments, the starting material is a partially refined Cannabis product such as hashish and hashish oil.

These methods also preferably comprise extracting from the plant part starting material at least one of tetrahydrocannabinol and cannabidiol, where contacting the plant part starting material with a fluid such as carbon dioxide or an organic alcohol. Without wishing to be bound to a particular theory, it is believed that tetrahydrocannabinol and cannabidiol are dissolved into the fluid upon said contacting. The compounds that are dissolved into the fluid are not limited to tetrahydrocannabinol and cannabidiol and may be other compounds present in the plant part starting materials. Organic alcohols suitable for fluid extract include, but are not limited to, methanol, ethanol, propanol, butanol, pentanol, hexanol, heptanol, octanol and isomers thereof. In preferred embodiments, the extracting stage is carried out after the selecting stage comprised above.

These methods also preferably comprise adjusting the concentration or amount of tetrahydrocannabinol and cannabidiol that results from the extracting stage described above. This adjusting stage can occur by any known suitable methods for adjusting the concentration of tetrahydrocannabinol and cannabidiol in mixtures, e.g., solutions and emulsions, that comprise tetrahydrocannabinol and cannabidiol.

These methods also preferably comprise adding the extract of tetrahydrocannabinol and cannabidiol having the adjusted concentration thereof obtained from the adjusting stage described above to a sublingual or buccal product, such as the lysophilisates described above, to introduce the tetrahydrocannabinol and/or cannabidiol into the sublingual or buccal product. The resulting sublingual or buccal products having the tetrahydrocannabinol and/or cannabidiol introduced therein are suitable for oral use.

Other embodiments of the present invention relate to a Cannabis derived botanical drug product formulated for sublingual delivery in aerosol or spray form, which offers unexpected advantages over known modes of cannabis delivery. The invention also relates to a device for delivering such a composition as an aerosol or spray.

Formulations according to the invention may include a propellant or may be dispensed using a pump spray device. The spray or aerosol devices may have upright or inverted valves. Furthermore, the aerosol or spray device may be adapted specifically for sublingual or pulmonary mucosal delivery. The device may also be adapted to dispense particles of a particular size, thereby optimizing the uptake.

It is known that sublingual and pulmonary mucosa delivery of a pharmaceutically active agent results in fast uptake. The active agent is administered to the sublingual or pulmonary mucosa, from which it is rapidly absorbed into the bloodstream.

Aerosol or spray delivery of a composition to the sublingual or pulmonary mucosa is particularly convenient and effective, and promotes fast-uptake.

In one of the preferred embodiments of the present invention, a composition suitable for aerosol delivery is provided comprising a Cannabis derived botanical drug product and a propellant. The propellant may be, for example, 1,1,1,2-tetrafluoroethane (HFC-134a), 1,1,1,2,3,3,3-heptafluoropropane (HFC-227) or butane. Most preferably, the propellant included in the composition is HFC-134a or HFC-227.

Preferably, the composition of the present invention includes a carrier. In a preferred embodiment of the invention, the carrier is a lower alkyl (C₁-C₄) alcohol, a polyol, or a (poly)alkoxy derivative. In embodiments, the carrier is a C₁-C₄ alkyl alcohol or a lanolin alcohol and, preferably, is ethanol or isopropyl alcohol. The most preferred alcohol is ethanol.

The preferred polyols include propylene glycol and glycerol and the preferred (poly)alkoxy derivatives include polyalkoxy alcohols, in particular 2-(2-ethoxyethoxy)ethanol (available under the Trademark TRANSCUTOL).

Further preferred (poly) alkoxy derivatives include polyoxyalkyl ethers and esters, such as polyoxyethylene ethers or esters. The preferred polyoxyethylene ethers and esters are polyoxyethylene alkyl ethers, polyoxyethylene castor oil derivatives, polyoxyethylene sorbitan fatty acid esters and polyoxyethylene stearates.

The preferred fatty acid alkyl esters are ethyl oleate, isopropyl myristate and isopropyl palmitate. The preferred polyalkylene glycol is polyethylene glycol.

In preferred embodiments, the inventive composition can comprise up to 50% or, preferably, 25% w/w carrier. More preferred embodiments include between 3% and 15% w/w, or between 4 and 10% w/w carrier. The pharmaceutical compositions can comprise between 50% and 99% w/w, preferably between 75% and 99% w/w, and, more preferably, between 88% and 95% w/w HFC-134a or HFC-227.

In further embodiments, compositions used in the present invention can comprise a plurality of different carriers.

Further excipients can be included in the formulations employed in the present invention. For example, neutral oils as well as surfactants (the latter for aiding the smooth operation of the valve), as are well known to those skilled in the art, may be included.

Thus, in further preferred embodiments, compositions employed in the invention can comprise an organic surfactant. The preferred organic surfactant is oleyl alcohol, although others can be employed, including sorbitan trioleate, sorbitan mono-oleate, sorbitan monolaurate, polyoxyethylene (20) sorbitan monolaurate, polyoxyethylene (20) sorbitan mono-oleate, natural lecithin, oleyl polyoxytheylene (2) ether, stearyl polyoxyethylene (2) ether, lauryl polyoxyethylene (4) ether, block copolymers of oxyethylene and oxypropylene, oleic acid, synthetic lecithin, diethylene glycol dioleate, tetrahydrofurfuryl oleate, ethyl oleate, isopropyl myristate, glyceryl mono-oleate, glyceryl monostearate, glyceryl monoricinoleate, cetyl alcohol, stearyl alcohol, cetyl pyridinium chloride, olive oil, glyceryl monolaurate, corn oil, cotton seed oil or sunflower seed oil.

It is preferable to include a flavouring oil in a formulation to be delivered sublingually. The preferred flavouring oil is peppermint oil, although it is clear that other flavour oils may be used, according to preference.

Some of the preferred compositions for the aerosol delivery according to the present invention contain tetrahydrocannabinols (THCs), such as delta-9tetrahydrocannabinol, one major active constituent of cannabis.

Many of the readily available substances derived from the cannabis plant are extracted in liquid form which may itself be directly sprayed using a pump spray or which may be soluble directly in the propellant, whilst other cannabis forms need to be solubilized in a co-solvent, such as ethanol, thus causing or allowing all or a proportion of the active agent present in the composition to dissolve and/or remain in solution, even after it has been dispensed.

The pharmaceutical compositions can be partial solutions in which only a proportion of the pharmaceutically active agent present therein is dissolved in the propellant and co-solvent, with the remainder being in suspension or suspendable. The exact proportions of dissolved and suspended active agent will depend upon the active agent concerned, its concentration and the identity and quantity of the co-solvent(s) used. In preferred embodiments the compositions are in the form of liquid solutions when maintained under pressure in devices in accordance with the invention.

In a particularly preferred embodiment of the invention the composition comprises a solution of delta-9-tetrahydrocannabinol in ethanol as a co-solvent and HFC-134a as a propellant.

The compositions of the present invention may also comprise cannabis in combination with other pharmaceutically active agents. For example, a formulation particularly suitable for providing improved anti-emetic effect comprises cannabis as the primary agent, with corticosteroid as a supplemental agent. In order to decrease toxicity of the primary agent, cannabis may be formulated together with the supplemental agent phenothiazine. Concurrent use of cannabis with proclorperazine in low doses can reduce incidence of dysphoria which can accompany the administration of cannabis.

According to a further aspect of the invention, devices for delivering the cannabis compositions of the first aspect of the invention are provided.

Devices for administering metered aerosol doses of pharmaceutical preparations are known and are not particularly limited so long as they meter the aerosol dose. The devices for administering the metered aerosol doses of the Cannabis derived botanical drug product described in US 2012/0328718 are incorporated herein by reference.

Another embodiment of the present invention relates to the form of the Cannabis derived botanical drug product, which is fast dissolve tablet or granule as described in Manufacture of Nanoparticulate Zydis Fast-Dissolve Tablets using Fish Gelatin by D. Bahl, I. Vitez, and K. Crowley, incorporated herein by reference. In these embodiments, the Cannabis derived botanical drug product, which includes at least one of THC and CBD, is incorporated into the fast dissolve tablet or granule. The methods of making the fast dissolve tablet or granule include those described in the D. Bahl et al.

Another embodiment of the present invention relates thin films or wafers that dissolve in the oral cavity as described in Wafers Technology—A Newer [Approach] to Smart Drug Delivery System, by Vibhooti and Preeti, Indian Journal of Research In Pharmacy and Biotechnology, Vol. 1(3), pp. 428-439, incorporated herein by reference. Specifically, the wafers comprise a dissolvable polymer such as at least one of sodium alginate, hydroxypropylmethyl cellulose, hydroxypropyl cellulose, hydroxyethyl cellulose, pectin, polyethylene oxide, and polyvinyl alcohol. In addition, the wafers can further comprise lactose or polystyrene. The wafers are usually obtained by methods comprising adding deionized water to a mixture of the dissolvable polymer, at least one of TCH and CBD, and a bulking agent such as lactose and mixing these components to form an aqueous formulation. The aqueous formulation is then subjected to freeze-drying and drying. The freeze-drying, which preferably occurs after the adding and before the drying, is carried out by placing the aqueous formulation in molds and placing the molds in a freeze dryer set to a temperature of from −100° C. to −10° C., most preferably from −80° C. to −30° C., and for example at −60° C. for a period of from 10 minutes to 5 hours, preferably 30 minutes to 4 hours, more preferably from 45 minutes to 3 hours, for example 2 hours. The drying is preferably carried out after the freeze drying and can occur at freeze-drying temperatures or at a temperature above that so long as the wafer does not melt. The pressure of the atmosphere for drying is atmospheric pressure or less than atmospheric pressure (eg. 760 Torr), such as 1 milliTorr to 100 milliTorr, for example 25 milliTorr. The time period for drying is not particularly limited so long as the wafer does not melt, such as 24 hours.

In all embodiments of the wafers, at least one of THC and CBD is present in the wafers in an amount of 1 to 30 milligrams per single dose. This value is either for THC or CBD when they are present by themselves or for the combination of THC and CBD when they are present together in the wafer.

In alternative embodiments to the wafer, the THC and/or CBD can be added to the wafer after it is produced from the drying phase. This addition of the THC and/or CBD to the wafer can occur by dissolving THC and/or CBD into a suitable solvent that is not an “organic solvent” as discussed above and applying the dissolved THC and/or CBD to the wafer. The dissolved THC and/or CBD can be in the form of, e.g., droplets when added to the wafer.

Additional embodiments of the present invention relate to the presence of at least one of THC and CBD in orally disintegrating tablets, such as those described in The Effect of Recent FDA Guidance on ODT Technologies and Applications by McGlaughlin et al. (Pharmaceutical Technology Supplement to the September 2009 issue), incorporated herein by reference.

Additional embodiments of the present invention relate to the presence of at least one of THC and CBD in orally dissolving strips, such as those described in A New Approach To Oral Drug Delivery System by Bala et al. (International Journal of Pharmaceutical Investigation, 2013 April-June, Vol. 3(2), 67-76), incorporated herein by reference.

Additional embodiments of the present invention relate to the presence of at least one of THC and CBD in buccoadhesive compositions, such as those described WO 2007/096906, incorporated herein by reference.

Additional embodiments of the present invention relate to the presence of at least one of THC and CBD in compositions used to deliver Vitamin B12, such as those described U.S. Pat. No. 8,609,630, incorporated herein by reference. In this instance the described approach is used to deliver THC, CBD and other cannabinoids.

Additional embodiments of the present invention relate to the presence of at least one of THC and CBD in compositions suitable for sublingual administration of THC and/or CBD, where the composition is in the form of a rapidly-disintegrating tablet as described in WO 2009/047321, incorporated herein by reference. The tablet comprises excipients capable of releasing carbon dioxide (CO₂) in the sublingual site and comprises a cyclodextrin compound. The excipients include, but are not limited to, a bicarbonate and an acid.

A suitable bicarbonate is sodium bicarbonate and a suitable acid is citric acid.

A cyclodextrin suitable for use in the present composition may be, for instance, either β-cyclodextrin or 2-hydroxypropyl-β-cyclodextrin; the composition of the invention preferably contains 2-hydroxypropyl-β-cyclodextrin.

The tablets are usually obtained by methods comprising: a) sieving the excipients and the raw material; b) mixing; c) compressing the mixture to produce said finished tablet.

The quantity of citric acid comprised within the tablets, for instance, is between 5 and 20% w/w of the total weight of the composition, and preferably amounts to 10% w/w.

The quantity of bicarbonate comprised within the tablets, for instance, is between 5 and 20% w/w of the total weight of the composition, and preferably amounts to 12% w/w.

The quantity of cyclodextrin contained in the composition shall preferably be between 27 and 40% w/w of the total weight of the composition.

Usually, when the tablets are placed inside the mouth, the tablets disintegrate between 60 and 120 seconds.

The excipients capable of releasing carbon dioxide (CO₂) in the sublingual site and comprises a cyclodextrin compound described herein can be incorporated into any of the embodiments described herein.

Additional embodiments of the present invention relate to the presence of at least one of THC and CBD in effervescent compositions suitable for sublingual administration of THC and/or CBD, the effervescent compositions described in U.S. Pat. No. 6,200,604, incorporated herein by reference. Usually, these effervescent compositions comprise effervescent enhancers that increase or accelerate oral drug absorption. Effervescent enhancers or “effervescent” or “effervescent agent(s)” can be used alone or in combination with other penetration enhancers, which leads to an increase in the rate and extent of absorption of THC and/or CBD.

Preferably, the effervescent is provided in an amount of between about 5% and about 95% by weight, based on the weight of the finished tablet, and more preferably in an amount of between about 30% and about 80% by weight. It is particularly preferred that sufficient effervescent material be provided such that the evolved gas is more than about 5 cm³ but less than about 30 cm³, upon exposure of the tablet to an aqueous environment. However, the amount of effervescent agent must be optimized for each specific drug.

The term “effervescent agent” includes compounds which evolve gas. The preferred effervescent agents evolve gas by means of a chemical reaction which takes place upon exposure of the effervescent agent (an effervescent couple) to water and/or to saliva in the mouth. This reaction is most often the result of the reaction of a soluble acid source and a source of carbon dioxide such as an alkaline carbonate or bicarbonate. The reaction of these two general compounds produces carbon dioxide gas upon contact with water or saliva. Such water-activated materials must be kept in a generally anhydrous state and with little or no absorbed moisture or in a stable hydrated form, since exposure to water will prematurely disintegrate the tablet. The acid sources may be any which are safe for human consumption and may generally include food acids, acid and hydrite antacids such as, for example: citric, tartaric, amalic, fumeric, adipic, and succinics. Carbonate sources include dry solid carbonate and bicarbonate salt such as, preferably, sodium bicarbonate, sodium carbonate, potassium bicarbonate and potassium carbonate, magnesium carbonate and the like. Reactants which evolve oxygen or other gasses and which are safe for human consumption are also included.

The effervescent agent(s) of the present invention is not always based upon a reaction which forms carbon dioxide. Reactants which evolve oxygen or other gasses which are safe for human consumption are also considered within the scope. Where the effervescent agent includes two mutually reactive components, such as an acid source and a carbonate source, it is preferred that both components react completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. However, in other embodiments of the present invention, the amount of either acid or carbonate source may exceed the amount of the other component. This may be useful to enhance taste and/or performance of a tablet containing an overage of either component. In this case, it is acceptable that the additional amount of either component may remain unreacted.

The present dosage forms may also include in amounts additional to that required for effervescence a pH adjusting substance. For drugs that are weakly acidic or weakly basic, the pH of the aqueous environment can influence the relative concentrations of the ionized and unionized forms of the drug present in solution according to the Henderson-Hasselbach equation. The pH solutions in which an effervescent couple has dissolved is slightly acidic due to the evolution of carbon dioxide. The pH of the local environment, e.g., saliva in immediate contact with the tablet and any drug that may have dissolved from it, may be adjusted by incorporating in the tablet a pH adjusting substances which permit the relative portions of the ionized and unionized forms of the drug to be controlled. In this way, the present dosage forms can be optimized for each specific drug. If the unionized drug is known or suspected to be absorbed through the cell membrane (transcellular absorption) it would be preferable to alter the pH of the local environment (within the limits tolerable to the subject) to a level that favors the unionized form of the drug. Conversely, if the ionized form is more readily dissolved the local environment should favor ionization.

The aqueous solubility of the drug should preferably not be compromised by the effervescent and pH adjusting substance, such that the dosage forms permit a sufficient concentration of the drug to be present in the unionized form. The percentage of the pH adjusting substance and/or effervescent should therefore be adjusted depending on the drug.

Other components described in U.S. Pat. No. 6,200,604 can be included in these compositions with the effervescent. Further, these compositions can be made according to the methods described in U.S. Pat. No. 6,200,604.

The effervescent agents described herein can be incorporated into any of the embodiments described herein.

In one embodiment, the at least one polyvinylpyrrolidone/vinyl acetate copolymer that forms an open matrix network and the at least one of tetrahydrocannabinol and cannabidiol present within the open matrix network comprise a composition that dissolves for instance substantially dissolved, substantially completely dissolved, completely dissolved, substantially liquid, etc.) in less than a minute (1 to 60 seconds e.g., 10 seconds, 20 seconds, 30 seconds, 40 seconds, 50 seconds) preferably within 30 seconds or upon contact with an aqueous solution or with saliva, and the composition is an orodispersible pharmaceutical dosage form.

The current invention describes formulation of multiple orodispersable formulations for instance, as described in J Nat Sci Biol Med. 2010 July-December; 1(1): 2-5. doi: 10.4103/0976-9668.71663 for the deliver for delivery of THC, CDB, Cannabis Botanical product, purified cannabinoid and synthesized cannabinoids for use in treatment of medical conditions and for recreational use. In addition the invention describes use of aerosols for delivery of Botanical Cannabis product, THC, CDB, isolated cannabinoids and synthetic cannabinoids by aerosol delivery to pulmonary mucosa Br J Clin Pharmacol. 2003 December; 56(6): 600-612. doi: 10.1046/j.1365-2125.2003.01893.x PMCID: PMC1884297.

We claim:

1. A composition comprising: (a) at least one polyvinylpyrrolidone/vinyl acetate copolymer that forms an open matrix network; and (b) at least one of tetrahydrocannabinol and cannabidiol present within the open matrix network.

2. The composition according to claim 1, wherein at least 80% of the composition dissolves within 30 seconds upon contact with an aqueous solution or with saliva, and the composition is an orodispersible pharmaceutical dosage form.

3. The composition according to claim 1, wherein tetrahydrocannabinol is present within the open matrix network.

4. The composition according to claim 1, wherein cannabidiol is present within the open matrix network.

5. The composition according to claim 1, wherein tetrahydrocannabinol and cannabidiol are present within the open matrix network.

6. The composition according to claim 1, wherein said least one of tetrahydrocannabinol and cannabidiol is present within the open matrix network in an amount of from 1 to 30 milligrams per single dose.

7. The composition according to claim 1, wherein tetrahydrocannabinol is present within the open matrix network and present in an amount of from 1 to 30 milligrams per single dose.

8. The composition according to claim 1, wherein cannabidiol is present within the open matrix network and present in an amount of from 1 to 30 milligrams per single dose.

9. The composition according to claim 1, wherein tetrahydrocannabinol and cannabidiol are present within the open matrix network and present in an amount of from 1 to 30 milligrams per single dose.

10. The composition according to claim 1, wherein said least one of tetrahydrocannabinol and cannabidiol is present within the open matrix network in an amount of from 5 to 10 milligrams per single dose.

11. The composition according to claim 1, wherein tetrahydrocannabinol is present within the open matrix network and present in an amount of from 5 to 10 milligrams per single dose.

12. The composition according to claim 1, wherein cannabidiol is present within the open matrix network and present in an amount of from 5 to 10 milligrams per single dose.

13. The composition according to claim 1, wherein tetrahydrocannabinol and cannabidiol are present within the open matrix network and present in an amount of from 5 to 10 milligrams per single dose.
